(19) United States
(12) Patent Application Publication
(10) Pub. No.: US 2010/0081608 A1
(43) Pub. Date: Apr. 1, 2010

(54) Use of Human Chorionic Gonadotropin (hCG) by Oral-Sublingual or Injectable Route as an Appetite-Suppressant Agent.

(76) Inventor: DANIEL OSCAR BELLUSCIO, BUENOS AIRES (AR)

Correspondence Address: Ignacio De Las Carreras, Talcahuano 958 - 18th. Floor, Apart "C", Buenos Aires, C1013AAT, AR

(21) Appl. No.: 12/430,936

(22) Filed: Apr. 28, 2009

(30) Foreign Application Priority Data
Apr. 28, 2008 (AR) 20080101788

(51) Int. Cl. A61K 38/24; A61P 3/04

(52) U.S. Cl. 514/8

(57) ABSTRACT

Use of Human Chorionic Gonadotropin (hCG) by oral-sublingual or injectable route for the treatment of several food disorders, as an appetite-suppressant agent, food compulsiveness as well as all of those pathologies related to hunger and/or appetite modifications, including overweight, obesity, anorexia, bulimia, emotional hyperphagia, without excluding other pathologies associated to overingestion or reduced ingestion. It also includes its use for the treatment of behavior disorders associated with an increased ingestion, either behavior disorders, neurosis, borderline personality disorders or psychosis, without excluding other psychosomatic disorders.

This invention is related to the “Use of Human Chorionic Gonadotropin (hCG) by Oral-Sublingual or Injectable Route as an Appetite Suppressant Agent”.

### SUMMARY

Use of Human Chorionic Gonadotropin (hCG) by oral-sublingual or injectable route for the treatment of several food disorders, as an appetite-suppressant agent, food compulsiveness as well as all of those pathologies related to hunger and/or appetite modifications, including overweight, obesity, anorexia, bulimia, emotional hyperphagia, without excluding other pathologies associated to overingestion or reduced ingestion. It also includes its use for the treatment of behavior disorders associated with an increased ingestion, either behavior disorders, neurosis, borderline personality disorders or psychosis, without excluding other psychosomatic disorders.

### APPLICATION FIELD

It is applicable in the treatment of pathologies of hunger and satiety mechanisms. Hunger, satiation and energetic balance are regulated by a redundant neuroendocrine system integrated at the hypothalamus level.

The system consists of a complex web of neurohormonal circuits that include short and long lasting molecular signals of central and peripherical origin as well as other sensory, mechanical and cognitive-type factors.

The system minimizes the impact of fluctuations of ingestion and energetic expense on the fatty mass and the body weight. The short-lasting signals, most of which are gastrointestinal tract hormones, regulate the amount of food consumed in each meal time. The long-lasting signals reflect the fat reserve size.

Endogenous opioids and hunger: The pro-opiomelanocortine (POMC) contains beta-endorphin, which in turn has met-encephalin and may also produce other shorter encephalins. In the brain, it is mainly located at the hypothalamic area.

The POMC is post-translationally modified thus giving rise to other biologically active peptides including ACTH, Beta-endorphins. These peptides exert their effect through melanocortine receptors (MCR), five of which have been described.

The MC3R and MC4R receptors are the ones involved in the regulation of hunger and satiation. Their stimulation has a central anorexigenic effect. Furthermore, they are thermogenesis mediators in the SNS, as a consequence of which they induce the loss of weight.

The MC4R is exclusively expressed in the neuroendocrine system and it is active in the areas that regulate the food ingestion, such as NPV, the dorsomedial hypothalamus and the lateral hypothalamic area.

The a-MSH is an agonist of MC3R and MC4R and, consequently, it is a very important anorexigenic signal. The expression of a-MSH is increased by the presence of leptine in the POMC neurons of the NAr, while at the same time it inhibits the AgRP neurons.

The role of opioids in the paraphysiology of food disorders would be explained through the self-addiction model. This model proposes that the exacerbated starvation by excessive exercise is itself a kind of body addiction to endogenous opioids.

We demonstrated in a series of studies that the administration of Human Chorionic Gonadotropin (hCG), which contains Beta-endorphin in its molecule, whether by oral-sublingual or intramuscular injectable route, has a remarkable appetite-suppressing action when administered according to the techniques described below. Its mechanism of action lies precisely at the hypothalamus level, wherein the hunger and satiation signals are generated.

### PROBLEM TO SOLVE

Food Disorder: Obesity and Overweight: The obesity and overweight are defined as an abnormal or excessive accumulation of fat which may be harmful to health.
Obesity and Overweight Details: The last calculations of the WHO indicate that in 2005 there were worldwide:
About 1600 million adults (older than 15 years old) suffering from overweight.
At least 400 million obese adults.

Furthermore, the WHO estimates that in 2015 there will be about 2300 million adults suffering from overweight and more than 700 million obese adults.

In 2005, there were at least 20 million 5-year-old infants suffering from overweight worldwide.

Although this issue used to be considered as problem exclusive of high-income countries, overweight and obesity are spectacularly increasing in low- and medium-income countries, mainly in the urban areas.

Which are the usual repercussions of overweight and obesity on health?

Overweight and obesity have serious consequences for health. The risk progressively increases as the BMI increases. The high BMI is an important risk factor for chronic diseases, such as the following:
Cardiovascular diseases (especially cardiopathies and cerebrovascular accidents), which are the main cause of death in the whole world, with 17 million yearly deaths.
Diabetes, which has rapidly become a world epidemic. The WHO estimates that the deaths from diabetes will rise by more than 50% in the next 10 years in the world.
Locomotive apparatus diseases and, particularly, arthrosis.
Some cancers, such as endometrial, breast and colon cancer.
   Food Disorder: Anorexia and Bulimia: Bulimia or nervosa bulimia (excessive hunger) is a mental disorder associated with food. The term “bulimia” comes from the Latin bulimia, which, in turn, comes from the Greek (boulimia) which, in turn, is composed of bous (ox) and limos (hunger).

Its essential characteristic is that an individual suffers from compulsive binge eating events followed by a feeling of guiltiness and control lost. It usually alternates with fast or very little food ingestion events but later on the individual suffers again from compulsive ingestion events.

Binge eating consists in eating in less than two hours an amount of food bigger than that which would be eaten by most individuals.

Despite the fact that the type of food eaten in this binge eating may be varied, generally it is sweets and food with a high caloric content, such as ice-cream, cakes or chocolate.

Another essential characteristic of this disorder relates to inappropriate compensatory behaviors to prevent weight gain. Most individuals use different procedures as an attempt to compensate for the binge eating, the most usual of which is the provocation of vomiting.

Food Disorder: Hyperphagia in other psychological alterations: Excessive ingestion as a reaction to stressing events which results in obesity. Mourning, grief, accidents, surgical procedures and emotionally stressing events may generate a “reactive obesity”, especially in diseased individuals predisposed to weight gain.
Inconvenients of appetite-suppressing medicaments: When prescribing it, it is necessary to bear in mind the following characteristics of the appetite-suppressing agents:
they are modestly effective in the ponderal reduction, thus causing a weight loss ranging from 8 to 10 Kg.
they have a high abuse, dependence and tolerance potential, and deprivation syndrome.
their main secondary effects appear at the following levels:
   CARDIOVASCULAR SYSTEM: palpitations, tachycardia, arterial hypertension, precordial color, arrhythmia.
   GASTROINTESTINTAL SYSTEM: mouth dryness, nausea, vomits, abdominal aches, diarrhea, constipation.
   CENTRAL NERVOUS SYSTEM: Overstimulation, excitement, insomnia, anguish, euphoria, depression, migraine, psychotic episodes, convulsions.

(Known) Appetite-Suppressant Medicaments
Generic name

| | |
|---|---|
| Noradrenergic agents | Benzphetamine |
| | Phendimetrazine |
| | Diethylpropion |
| | Phentermine |
| | Mazindol |
| | Phenylpropanolamine |
| Serotoninergic agents | Phenfluramine |
| | Dexphenfluramine |
| | Fluoxetine |
| | Ephedrine/caffeine |

### SOLUTION PROVIDED BY THE INVENTION

Human Chorionic Gonadotropin (hCG) Description: The human Chorionic Gonadotropin (hCG) is a glucoprotein and represents the association between an α (alpha) chain and a β (Beta) chain. The hCG is obtained from the urine of pregnant women and it is not homogeneous.

Highly purified medicaments also contain several moieties differing from sialic acid and in the biological action. The amount of hCG is indicated in biological action units.

The hCG hormonal effect is based on its ability to stimulate the biosynthesis of sexual steroids in the gonads (ovaries and testes). The hCG action is qualitatively the same as that of the pituitary gonadotropine (LH). However, the hCG has a considerably longer half-life, which leads to a stronger action in case of a cumulative administration.

In the ovaries, the hCG stimulates the granulosa, theca and stroma or luteal cells in order to keep the progesterone and estradiol production.

In granulosa cells of the small follicles, the estradiol biosynthesis is preferably stimulated by high doses of hCG. As in the granulosa cells of the dominant mature follicles and/or luteinizing granulosa cells, the progesterone biosynthesis is stimulated by high hCG doses. Likewise, the hCG stimulates the production of biologically active peptides in the ovary, said peptides being important for the reproduction regulation (for example, inhibition, relaxation, plasminogen-activator-inhibitor).

In Leydig cells, the hCG stimulates the production of testosterone and other sexual steroids, such as dihydrotestosterone, 17 OH-progesterone and estradiol.

Although the primary prescription of hCG is related to the infertility area, different performed researches that have been carried out conclude that it may be successfully used in a very different diseases without undesired effects since it is a natural-source medicament.

In the traditional Pharmacopoeia, the Chorionic Gonadotropin prescription is applied only through the intramuscular injectable route. The novelty in this invention is that it enables its administration both by intramuscular and oral-sublingual route as an appetite-suppressant agent (a non-foreseen indication before this invention), thus avoiding all of the inconveniences derived from the administration of drugs with potential secondary effects. The presence of Beta endorphine in the hCG molecule would be responsible for the observed clinical phenomena.

### ADVANTAGES

Administration of hCG by oral-sublingual or intramuscular injectable route: Unlike the usual appetite-suppressant medicaments, the administration of hCG has no risks for patients since it lacks all of the characteristic side effects of appetite-suppressant medicaments. It does not have side effects or contraindications. The appetite suppression is highly significant.

### BRIEF DESCRIPTION OF THE DRAWINGS

In order to achieve the advantages herein briefly commented, to which the users and skilled persons in the art may add many others, there follows a description of the drawings that schematically illustrate the benefits of this invention without a determined scale in the accompanying sheets, wherein:

FIG. 1 is a drawing representing the transgression to the diet during the observation period. Gi: Initial gonadotropin (treatment beginning), Gf: Final Gonadotropin (end of treatment).

FIG. 2 is a drawing related to eating due to anxiety during the observation period. Gi: Initial gonadotropin (treatment beginning), Gf: Final Gonadotropin (end of treatment).

FIG. 3 is drawing related to the tiredness when getting up during the observation period. Gi: Initial gonadotropin (treatment beginning), Gf: Final gonadotropin (end of treatment).

FIG. 4 is a drawing related to the physical hunger during the observation period. Gi (treatment beginning). Gf: Final gonadotropin (end of treatment).

FIG. 5 is a drawing related to irritability during the observation period. Gi: Initial gonadotropin (treatment beginning), Gf: Final gonadotropin (end of treatment).

FIG. 6 is a drawing related to eating when not being hungry during the observation period. Gi: Initial gonadotropin (treatment beginning), Gf: Final gonadotropin (end of treatment).

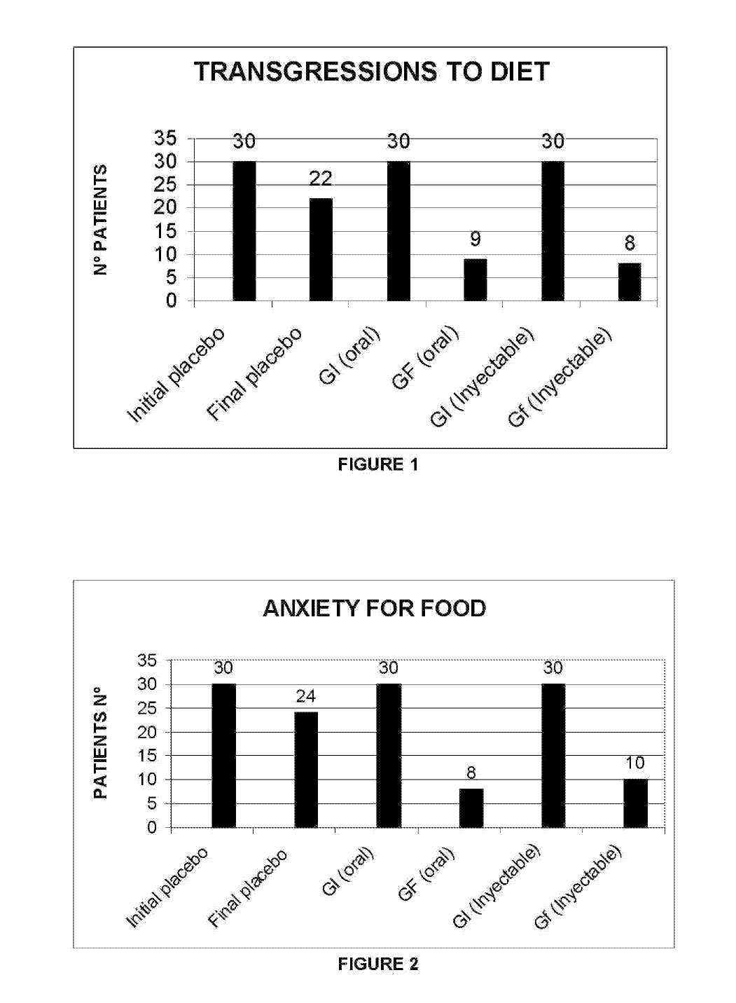

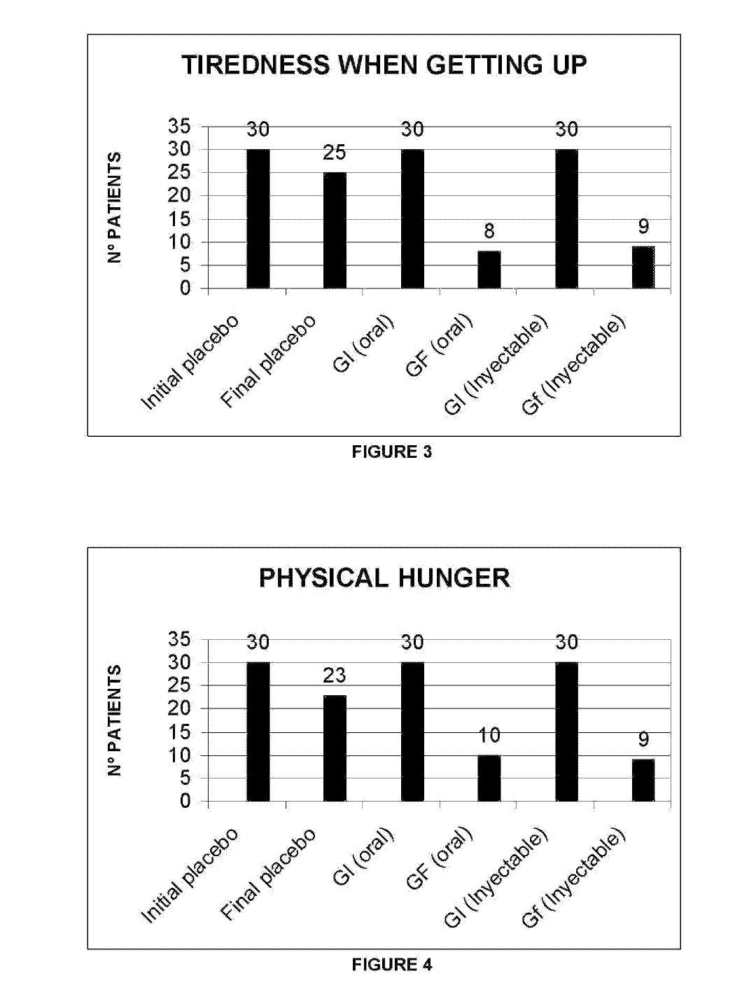

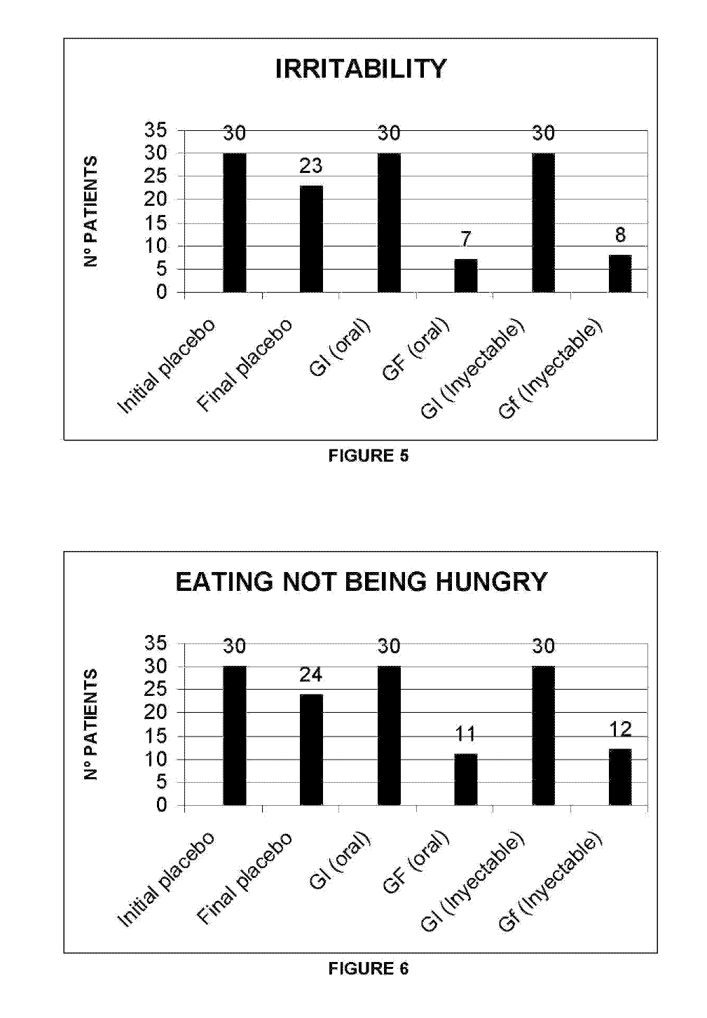

Clinical Experiences: Ninety patients suffering from varied food disorders were studied, most of them stated to have a daily overingestion with the subsequent overweight development.

The performed study was a double-blind type study for a five-week period. The placebo group (30 patients) received a saline solution, whereas the hCG group received hCG by oral route (from 200 to 500 international units daily, 30 patients) or intramuscular injectable route (from 130 to 200 IU, 30 patients).

In overweight or obesity cases, a very low calorie diet was prescribed to contribute with the body mass reduction.

Results: After revieweing the charts between the patients treated with Gonadotropin, whether by injectable or oral route, and the volunteers to whom the placebo was administered, the following parameters have shown significant differences from the statistical point of view regarding:

1. Physical hunger

2. Transgressions to the diet

3. Eating related to anxiety

4. Tiredness when getting up during the treatment period

5. Irritability during the treatment period

6. Eating without being hungry during the treatment period.

In all of the studied cases, patients have stated to feel clinically well during the research period.

The administration by injectable or oral route of hCG provides, through hypothalamic mechanisms:

1. Appetite reduction, better control over ingestions

2. Reduction of anxiety for food

3. Patient were in very good mood despite the fact of being subjected to a low-calorie diet

4. Overweight or obesity reduction, especially around the waist (central obesity) and abdomen

5. Reduction of cholesterol figures

6. Clinical improvement of diabetes type 2 or resistance to insulin

7. Feeling of wellbeing during the treatment period

8. Improvement in high blood pressure

### DESCRIPTION

Under medical supervision, the patient is administered Chorionic Gonadotropin (hCG) by the oral or injectable route. The daily Gonadotropin doses are adjusted between 300 to 500 international (oral-sublingual) units daily or 100 to 300 (injectable) IU during the treatment period.

Since most of these patients resort to appetite-suppressant medicaments because they display some degree of overweight or obesity, in such cases they are also prescribed a very low-calorie (about 500 Kcal/day), low-fat, hypohydrocarbonated, normoproteic diet, providing 200 grams of proteins from animal plus a combination of vegetables and carbohydrates until the indicated amount of calories is reached.

Use: The treatment is carried out for a period not less than a month and it may be extended up to two months. After that, a weight maintenance is indicated for a one month period, after that as of which it may be repeated again.

During the intervals, no treatment with hCG is indicated made and a usual hypohydrocarbonated diet is prescribed.

From the above description and the accompanying drawings, the constructive and functional advantages that characterize the claimed invention are clearly noticed and it is therefore considered as an advantageous technology improvement.

1. Use of human chorionic gonadotropin (hcg) by oral-sublingual or injectable route as an appetite-suppresant agent, characterized by the daily Human Chorionic Gonadotropin doses between 300 and 500 IU daily (International Units) (oral-sublingual) or 100 to 300 IU (injectable) during the treatment period.

2. Use of human chorionic gonadotropin (hcg) by oral-sublingual or injectable route as an appetite-suppresant agent, as the one claimed in claim 1 characterized by the administration of a very low-calorie (furnishing about 500 Kcal/day), low-fat, hypohydrocarbonated, normoproteic diet, providing 200 grams of animal proteins plus a combination of vegetables and carbohydrates until the indicated amount of calories is reached, for a period not less than a month, which may be extended up to two months, and can be repeated again. During pauses no treatment with hCG is indicated and an usual hypohydrocarbonated diet is prescribed.

3. Use of human chorionic gonadotropin (hcg) by oral-sublingual or injectable route as an appetite-suppresant agent, in accordance with claim 1, characterized by the use of the human Chorionic Gonadotropin (hCG) by oral-sublingual or injectable route for the treatment of different food disorders, as an appetite-suppressant agent, and food compulsiveness.

4. Use of human chorionic gonadotropin (hcg) by oral-sublingual or injectable route as an appetite-suppresant agent, in accordance with claim 1, characterized by the use of hCG for the treatment of all of those pathologies which disorders are hunger and/or appetite alterations, including overweight, obesity, anorexias, bulimias, emotional hyperphagias, without excluding other pathologies associated with overingestion or reduced ingestion.

5. Use of human chorionic gonadotropin (hcg) by oral-sublingual or injectable route as an appetite-suppresant agent, in accordance with claim one, characterized by the use of hCG for the treatment of behavior disorders associated with an increase in ingestion, whether they are behavior disorders, neurosis, borderline personality disorders or psychosis, without excluding other psychosomatic disorders.
